(19) United States
(12) Patent Application Publication
(10) Pub. No.: US 2022/0152327 A1
(43) Pub. Date: May 19, 2022

(54) PREDICTION AND AVOIDANCE OF ADVERSE OXYGEN DESATURATION EVENTS IN PATIENTS RECEIVING SUPPLEMENTAL OXYGEN

(71) Applicants: The Research Foundation for The State University of New York, Albany, NY (US); Neektec, LLC., Farmingville, NY (US)

(72) Inventors: John Brittelli, East Patchogue, NY (US); Dante T. Vigliotti, Farmingville, NY (US); Michael A. Kamchu, Farmingville, NY (US)

(73) Assignees: The Research Foundation for The State University of New York, Albany, NY (US); Neektec, LLC., Farmingville, NY (US)

(21) Appl. No.: 17/527,299

(22) Filed: Nov. 16, 2021

Related U.S. Application Data
(60) Provisional application No. 63/114,555, filed on Nov. 17, 2020.

(51) Int. Cl. A61M 16/00; A61M 16/10; G16H 40/63; G16H 50/20

(52) U.S. Cl. CPC A61M 16/026; A61M 16/101; A61M 16/0051; G16H 40/63; G16H 50/20; A61M 2230/63; A61M 2205/50; A61M 2230/40; A61M 2230/06; A61M 2230/20; A61M 2202/0208

(57) ABSTRACT

The present disclosure is directed to systems and methods of controlling supplemental oxygen supply devices, the methods include: receiving data from one or more sensors of a patient device operably connected to a patient; using the data as an input to a machine learning model that predicts whether or not a change to a supply amount of an oxygen enriched gas from the supplemental oxygen supply device is to be made; and transmitting a control signal to the supplemental oxygen supply device based on the predicted change.

### CROSS REFERENCE TO RELATED APPLICATION

This application claims priority from U.S. Provisional Application No. 63/114,555 filed Nov. 17, 2020, the entire contents of which are incorporated herein by reference.

### FIELD OF THE DISCLOSURE

The present disclosure is directed to devices and methods to be used with supplemental respiratory oxygen supplies and systems that maintain sufficient blood oxygen saturation in sub-acute patients receiving supplemental oxygen by controlling the dosing of oxygen with a measured response to the patient's actual blood oxygen saturation levels. This control of oxygen flow for patients in need of supplemental oxygen including, for example, chronic obstructive pulmonary disorder (COPD) patients, provides for sufficient blood oxygen levels for the patient, and avoids and/or reduces hypoxic events. Chronic Asthma, Emphysema, Congestive Heart Failure and several other cardio-pulmonary conditions are also addressed with supplemental oxygen. The present methods, systems and devices may also be used in subjects (e.g., obese individuals), who may also require supplemental oxygen, for example, to maintain elevated activity levels.

### BACKGROUND OF THE DISCLOSURE

For administration of oxygen therapy, oxygen can be delivered directly into the nostrils of the patient through a nasal cannula or a mask the patient wears. The cannula or mask are connected via a supply hose to a source of oxygen, such as an oxygen concentrator, liquid oxygen dewar or high pressure gas cylinder. The oxygen is typically delivered continuously or in pulse dose to the patient at a rate prescribed by a physician.

Specifically regarding oxygen concentrators, oxygen concentrators take advantage of pressure swing adsorption (PSA). Pressure swing adsorption involves using a compressor to increase gas pressure inside a canister that contains particles of a gas separation adsorbent. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. Published Patent Application No. 2009/0065007, entitled “Oxygen Concentrator Apparatus and Method”, which is incorporated in its entirety herein by reference.

Ambient air is composed of approximately 78% nitrogen and 21% oxygen with the balance comprised of argon, carbon dioxide, water vapor and other trace elements. If a gas mixture such as air, for example, is passed under pressure through a vessel containing a gas separation adsorbent bed that attracts nitrogen more strongly than it does oxygen, part or all of the nitrogen will stay in the bed, and the gas coming out of the vessel will be enriched in oxygen. When the bed reaches the end of its capacity to adsorb nitrogen, it can be regenerated by reducing the pressure, thereby releasing the adsorbed nitrogen. It is then ready for another cycle of producing oxygen enriched air. By alternating canisters in a two-canister system, one canister can be collecting oxygen while the other canister is being purged (resulting in a continuous separation of the oxygen from the nitrogen). In this manner, oxygen can be accumulated out of the air for a variety of uses including, but not limited to, providing supplemental oxygen to patients.

The average patient prescription is typically 2 liters per minute of highly concentrated oxygen, raising the overall oxygen level inhaled by the patient from the usual 21% in the atmosphere to a range of about 28% to about 35%, or higher. Although the average required oxygen flow rate is 2 liters per minute, the average oxygen concentrator is capable of at least 4-6 liters per minute, or more, or about 5 liters per minute.

Historically, hypoxia has been treated after a decrease in oxygen saturation levels occur. This has the potential to allow for the resistance of blood flow through the lungs to become increased, which is a known cause of cor pulmonale. Typically, treatment of hypoxia only occurs after an unsafe reduction in oxygen concentration is detected, thus not avoiding the onset of the hypoxic condition in the first place.

Further, the ability to control sources of oxygen, such as oxygen concentrators in an intelligent manner is typically limited. Oxygen supply from standard oxygen concentrators is limited to the manual setting of a supply amount or a manually programmed schedule that varies supply settings over the course of time. Thus, there is a need for more effective approaches for controlling oxygen supply.

What is desired is a system, devices, and method to preemptively avoid the occurrence of a hypoxic condition by modifying oxygen flow. Embodiments of the present disclosure provide devices and methods that address the above needs.

### SUMMARY OF THE DISCLOSURE

The present disclosure is directed to systems and methods of controlling supplemental oxygen supply devices, the methods comprising: receiving data from one or more sensors of a patient device operably connected to a patient; using the data as an input to a machine learning model that predicts whether or not a change to a supply amount of an oxygen enriched gas from a supplemental oxygen supply device is to be made; and transmitting a control signal to the supplemental oxygen supply device based on the predicted change.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 is a block diagram of a system for controlling a supplemental oxygen supply device, according to one embodiment of the disclosure.

FIGS. 2A and 2B are a diagram illustrating a high-level flow for controlling a supplemental oxygen supply device, according to an embodiment of the disclosure.

FIG. 3 is a flow diagram illustrating training and operation of a machine learning model, according to an embodiment of the disclosure.

FIG. 4 is a block diagram of another system for controlling a supplemental oxygen supply device, according to an embodiment of the disclosure.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of +0.1%, for other elements, the term “about” can refer to a variation of ±1% or ±10%, or any point therein.

As used herein, the term “substantially”, or “substantial”, is a broad term and is used in its ordinary sense, including, without limitation, being largely but not necessarily wholly that which is specified, which is equally applicable when used in a negative connotation to refer to the complete or near complete lack of an action, characteristic, property, state, structure, item, or result. For example, a surface that is “substantially” flat would mean either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element and a second element are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

As used herein, the terms “supplemental oxygen” and “supplemental respiratory oxygen” refer to oxygen delivered to patients in addition to the oxygen received by the patient through the inspiration of room or ambient air. Because room air contains some oxygen, the supplemental oxygen is provided in addition to the oxygen that would normally be inspired by the patient.

As used herein, the term “blood oxygen content” and “blood oxygen saturation” will typically be used to refer to blood oxygen saturation as commonly measured by the percentage of oxygen-saturated hemoglobin (SpO₂) although it can also refer to any suitable measurement for determining the level of oxygenation in a patient's blood. For example, it will be understood that blood oxygen content can be measured by a CO-oximeter. Furthermore, blood oxygen content can be estimated based on the partial pressures of oxygen (PaO₂) at various atmospheric pressures.

As used herein, the term “patient” means a mammal wherein supplemental oxygen is beneficial, such as a patient with restrictive and/or obstructive lung conditions, diseases, and/or diagnoses. In one embodiment the patient is a human. In certain embodiments, the patient has been diagnosed with a restrictive lung disorder such as, for example, bronchitis, emphysema, asthma, chronic asthma, congestive heart failure, and/or chronic obstructive pulmonary disease (COPD). In one embodiment, the patient has COPD. In some embodiments, the patient with COPD exhibits one or more of the following conditions: bronchitis, emphysema and asthma.

The patient can have SpO₂ levels below a typical range for a healthy patient, such as, for example, having a SpO₂ level of about 96% or less, about 95% or less, about 94% or less, about 93% or less, about 92% or less, about 91% or less, about 90% or less, about 89% or less, about 88% or less, about 87% or less, about 86% or less, about 85% or less, about 84% or less, about 83% or less, about 82% or less, about 81% or less, about 80% or less, about 79% or less, about 78% or less, about 77% or less, about 76% or less, about 75% or less, about 74% or less, about 73% or less, about 72% or less, about 71% or less, or about 70% or less.

As used herein, the term “care” refers to care provided to patients in hospital, mobile and/or residential settings. “Residential” can include, e.g., homes and long-term care facilities (such as nursing homes). “Care” also includes mobile care delivered in ambulatory situations, for example, when the patient is engaged in normal activities outside of his or her residence, such as shopping, attending concerts or other events, and traveling to appointments with health care professionals.

As used herein, the terms “continuous” and “continuously” (when referring to the measuring of blood oxygen content levels) mean that the blood oxygen content level of the patient will be measured without cessation or at intervals (fixed or variable) that are sufficiently small to still continue with the disclosed methods as discussed herein.

As used herein, the term “oxygen enriched gas” refers to a gas composed of at least about 50% oxygen or more, at least about 60% oxygen or more, at least about 70% oxygen or more, at least about 80% oxygen or more, at least about 85% oxygen or more, at least about 86% oxygen or more, at least about 87% oxygen or more, at least about 88% oxygen or more, at least about 89% oxygen or more, at least about 90% oxygen or more, at least about 91% oxygen or more, at least about 92% oxygen or more, at least about 93% oxygen or more, at least about 94% oxygen or more, at least about 95% oxygen or more, at least about 96% oxygen or more, at least about 97% oxygen or more, at least about 98% oxygen or more, at least about 99% oxygen or more, or a gas composed of between about 50% and about 99% oxygen, or between about 60% and about 98%, or about 70% and about 97%, or about 80% to about 96%, or about 90% to about 95%, or about 92% to about 94%.

This oxygen enriched gas can be delivered in a continuous flow or a pulse flow from an oxygen concentrator. A continuous flow is considered to be a supply of oxygen enriched gas being supplied to the patient throughout a majority of or the entirety of the respiratory cycle (both inspiration and expiration). A pulse flow is considered to be a supply of oxygen enriched gas such that the oxygen enriched gas in is supplied only at or near a beginning of the patient's inspiration portion of the respiratory cycle. Pulse flow typically decreases the overall supply of the oxygen enriched gas, as compared to continuous flow, and can obtain about the same therapeutic affect for the patient.

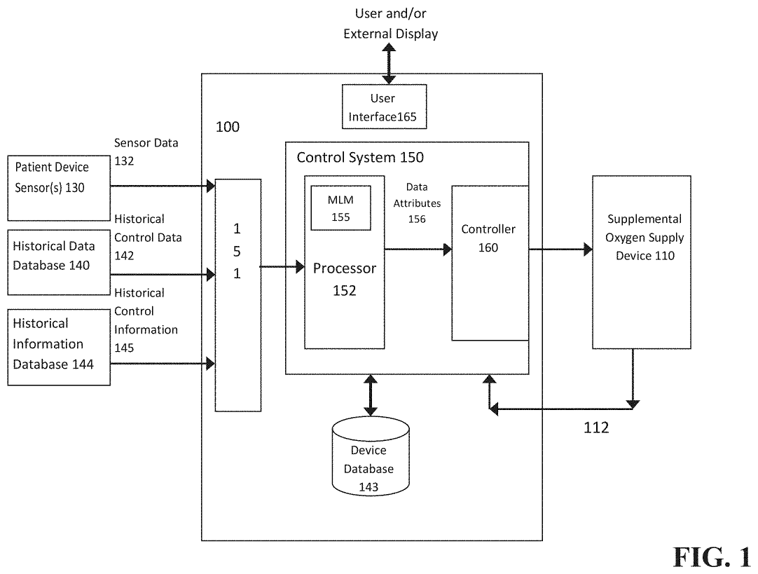

FIG. 1 is a block diagram of a system 100 for controlling a supplemental oxygen supply device 110 according to one embodiment of the present disclosure. The supplemental oxygen supply device 110 is configured to maintain, decrease, or increase a flow of oxygen enriched gas to a patient.

The supplemental oxygen supply device 110 can be any device able to provide oxygen at a higher percentage than atmospheric oxygen, such as an oxygen concentrator, liquid oxygen dewar or high pressure gas cylinder. In a specific embodiment, the supplemental oxygen supply device is an oxygen concentrator.

The system 100 includes a data interface 151 and control system 150. The control system includes a processor 152, which includes a machine learning model (MLM) 155, and a controller 160. As used herein, the term “machine learning model” is meant to include a single machine learning model or an ensemble of machine learning models. Each model in the ensemble may be trained to infer different attributes. The MLM 155 is a program module of the processor 152 that performs the methods and functions described herein. The MLM 155 can be programmed into the integrated circuits of the processor 152.

The data interface 151 receives various input data, which are processed, at least in part by the machine learning model (MLM 155). The results output by the MLM 155 are data attributes 156, which are received as input by the controller 160, which controls the environmental supplemental oxygen supply device 110 accordingly. Although FIG. 1 illustrates the system 100 being separate from the supplemental oxygen supply device 110, the system 100 can be incorporated within a housing and/or structure of the oxygen supply device 110, or the system 100 can transmit signals through any wired or wireless transmission modality.

The processor 152 can be a CPU. The processor 152 can be a single core or a multiple core processor. In other aspects, some of the processor 152 can be a graphics processing unit (GPU). In other aspects, the processor 152 can be an integrated circuit, application specific integrated circuit (ASIC), programmable logic device (PLD), digital signal processor (DSP), field programmable gate array (FPGA), logic gate, register, semiconductor device, chip, microchips, chip set, and so forth. When multiple processors 100 are used, the different processors can be of a different type. For example, one processor may be a CPU and another processor may be a GPU or an ASIC. In other embodiments using multiple processors 100, each of the multiple processors can be the same type of processor such as, for example, two or more CPU's.

Although not shown, system 100 can also include an electronic storage device/memory. The electronic storage device (as well as any database referred to herein) can include computer system readable media in the form of volatile memory, such as random access memory (RAM) and/or cache memory or others. In some embodiments, multiple electronic storage devices may be used. The electronic storage device can be any type of integrated circuit or other storage device adapted for storing data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

The control system 150 can receive various types of inputs, and from various sources. This includes sensor data 132, captured by patient device sensor(s) 130, historical control data 142 received from their storage locations in a historical data database 140, and historical information 145 received from their storage locations in a historical information database 144.

The sensor data 132 captured by patient device sensor(s) 130 can include one or more types of data captured by one or more types of sensors, which are included in one or more devices operably connected to the patient. These sensors include one or more of a heart rate sensor (which provides the patient's heart rate data), an accelerometer (which provides the patient's movement data and/or speed data in one or more axes), a barometer (which provides the patient's barometric pressure data (or pressurized equivalent altitude, such as in an airplane) and a SpO₂ sensor (which provides the patient's percentage of oxygen-saturated hemoglobin data).

The barometer data is used by system 100 for an altitude calculation and air pressure/density determination. This data in combination with the SpO₂ is used to maintain a partial pressure of oxygen (PAO₂) similar to what it would be at sea level, where availability of oxygen is the highest. PAO₂ is given by the Alveolar Gas Equation:

PAO₂=(Pₐₜₘ−PH₂O)FiO₂−PaCO₂/RQ whereby
PAO₂ is the partial pressure of oxygen in alveoli;
Pₐₜₘ is the atmospheric pressure;
FiO2 is the fraction of inspired oxygen;
PH₂O is the partial pressure of water;
PaCO₂ is the partial pressure of CO₂ in alveoli; and
RQ is the respiratory quotient,

FiO₂ (adjusted variable), PAO₂ (response variable), and Pₐₜₘ are focuses. All of the other variables are treated as constants. So, as atmospheric pressure decreases, in order to maintain a stable PAO₂, the FiO₂ is increased by increasing the liter flow of oxygen from the supplemental oxygen supply device 110.

Each of these sensors can be included in a single, wearable device for the patient to wear on any suitable part of their body. Also, these sensors can capture data continuously, or they can be configured to capture data every “N” seconds or “N” minutes. Further, each of the sensors can transmit sensor data 132 in any suitable way, such as through one or more of a wired internet connection, a wireless internet connection, a cellular connection, a Bluetooth connection, a Near Field Communication connection, etc.

The historical control data 142 received from storage locations in the historical data database 140 can be any previously captured data by the patient device sensor(s) 130 from the specific patient, and its associated control instruction sent to the supplemental oxygen supply device 110. This previously captured data can be recent, for example, from the past several hours or days, or this previously captured data can be more distant, for example from the past several weeks or months.

The historical information 145 received from storage locations in the historical information database 144 can be previously captured data by patient device sensor(s) 130 from patients other than the specific patient, and its associated control instruction sent to supplemental oxygen supply devices. This historical information database 144 can include all captured data from all patients, or the historical information database 144 can be configured to provide data for other patients that are similarly situated such as, for example, being the same/similar age, same sex, and same/similar respiratory issue/disease. This previously captured data in the historical information database can be recent, for example, from the past several hours or days, or this previously captured data can be more distant, for example, from the past several weeks or months.

Historical data database 140 and historical information database 144 can be local databases within system 100 and/or within the structure of the supplemental oxygen supply device. One or both of the historical data database 140 and historical information database 144 can be a remote or cloud based database, which is accessible by system 100 through a network connection.

In FIG. 1, the control system 150 also receives information from the supplemental oxygen supply device 110. The supplemental oxygen supply device 110 may provide data 112 about its operation, for example, settings and rate of operation over time, status of the supplemental oxygen supply device, and log files and errors/alerts.

Device database 143 contains profile information for the supplemental oxygen supply device 110. This profile information can include gas flow capabilities, and oxygen enrichment capabilities of the specific supplemental oxygen supply device 110.

The control system 150 receives these different data, processes them in the processor 152 them and controls, with the controller 160 the supplemental oxygen supply device 110 accordingly.

The MLM 155 can be useful to predict data attributes that can be difficult or cumbersome to develop using more conventional approaches. For example, the sensor data 132 may be used as input to the MLM 155, which then predicts various data attributes 156. The controller 160 then controls the supplemental oxygen supply device 110 according to these attributes. One example is that the MLM 155 may predict an increase of 1.5 liters/minute of oxygen enriched gas from the supplemental oxygen supply device 110 upon receipt of sensor data 132 indicating that the patient has stood up from a seated position.

The use of machine learning is especially beneficial for situations where the predicted attribute is a complex function of two or more factors, or when there is a desire for the system to self-learn or self-monitor certain relationships. For example, if the sensor data 132 indicates that the patient has stood up from a seated position, and that the barometric pressure is that of a pressurized airplane. Machine learning approaches can be used to learn these complex relationships for each specific patient. In addition, these complex relationships may change over time as the patient's health improves or degrades. Even if it were possible to expressly construct a model to regulate a supplemental oxygen supply device, it is desirable for machine learning techniques to automatically adapt to changes over time rather than manually changing the model to account for these shifts.

Returning to FIG. 1, the system 100 also includes a user interface 165. The user interface 165 provides an interface to the system 100, allowing an operator to monitor in real-time the supplemental oxygen supply device 110 and/or sensor data 132, and/or to review historical performance and/or to predict future performance. Through the user interface 165, the operator can also make changes to the device database 143. It may also allow the operator to configure different data inputs 132, 142, 145. Alternatively, or in addition to, the user interface 165 can transmit viewable data to an external display, such as one on the supplemental oxygen supply device 110, or an external display not physically connected to the supplemental oxygen supply device 110, such as a separate monitor and/or a mobile electronic device such as a mobile phone or tablet.

Alternatively, or in addition to, the user interface 165 can transmit an alert signal to an external server. This alert signal can be based on sensor data 132 indicating a dangerous and/or important condition such as blood oxygenation below a threshold, or lack of motion of the patient over a period of time, or heart rate below a certain threshold. This external server can be configured to receive such an alert signal, and then automatically transmit it to one or more mobile devices such as, for example, a mobile device of a medical professional, the patient, a caregiver and/or a family member.

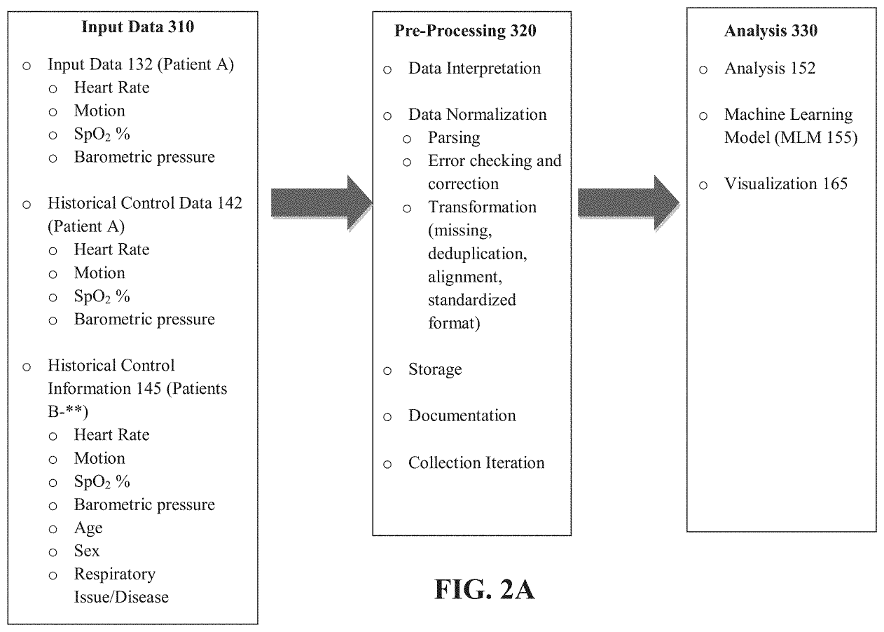

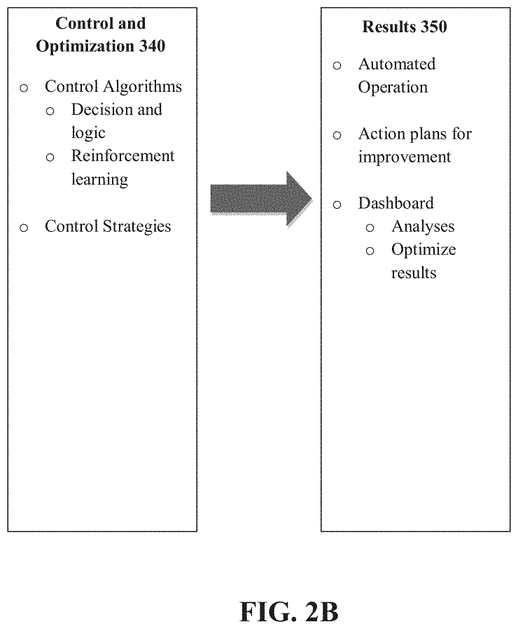

FIGS. 2A and 2B are a diagram illustrating a high-level flow for controlling the supplemental oxygen supply device 110, according to one embodiment of the disclosure. FIG. 2B is a continuation of FIG. 2A. Whereas FIG. 1 illustrates control concepts in the form of a system block diagram, FIGS. 2A and 2B organizes these concepts as a flow of data, actions and results. The input data 310 in FIG. 2A correspond to the inputs to the control system 150 in FIG. 1. The input data 310 includes sensor data 132 (which can include one or more of patient A's heart rate, motion, SpO₂% and barometric pressure), historical control data 142 (which can include historical measurements from one or more of patient A's heart rate, motion, SpO₂% and barometric pressure), and historical control information 145 (which can include historical measurements from one or more other patients B-**, and their historical patient A's heart rate, motion, SpO₂% and barometric pressure, as well as their associated ages, sex, and respiratory issue/disease). FIG. 2A lists examples of each of these categories, which are also described with respect to FIG. 1.

The input data 310 is pre-processed 320. This can include data interpretation and data normalization. Examples of normalization include, for example, parsing data, error checking and correction, and transformation. Missing data may be retrieved or noted as missing. Duplicate data may be de-duplicated or “de-duped”, i.e., duplicate data points are removed. Data from different sources may be aligned in time or space. Data may be reformatted to standardized formats used in further processing. Pre-processing 320 may also include data storage (e.g., in a memory of the system 100 and/or an external database), documentation and collection iteration. Documentation is the process of documenting the context of data, collection methodology, structure, organization, descriptions of variables and metadata elements, codes, acronyms, formats, software used, access and use conditions, and the like. Collection iteration is the process of iteratively collecting new forms of data and/or improving previous data collection procedures to improve data quality.

Pre-processed data is analyzed 330. Analytics performed by processor 152 can be performed for purposes of controlling the supplemental oxygen supply device 110 or for purposes of analyzing the supplemental oxygen supply device 110. Analysis can identify various patterns, as well as identifying areas of waste or potential improvement. As described above, MLM 155 is especially useful to learn complex relationships and/or to automatically adapt to changes.

Visualization of analysis results can be presented by the user interface 165. This user interface 165 can be configured to display various data and responses over time, as well as, for example, error alerts.

Continuing to FIG. 2B, based on the analysis 330, different types of control and optimization 340 can be implemented. For more traditional control algorithms, the control is defined by a set of control logic or rules. Reinforcement learning can be used to adapt control strategies over time. FIG. 2B also lists some specific control strategies, such as automatic increases of oxygen enriched gas flow above a step-wise increase. Control and optimization may be performed based on machine learning results. For example, how large the automatic increase of oxygen should be based on variations of input data 132 and previous variations and responses in historical control data 142 may be learned through machine learning analysis.

Box 350 lists some of the results and benefits that may be achieved. Improved control can result in avoidance of oxygen desaturation events, and more patient comfort. Automatic discovery of patterns and adaptation can result in a more automated operation of the supplemental oxygen supply device 110. It may also be useful to produce a dashboard that gives an overview of operation of the supplemental oxygen supply device 110.

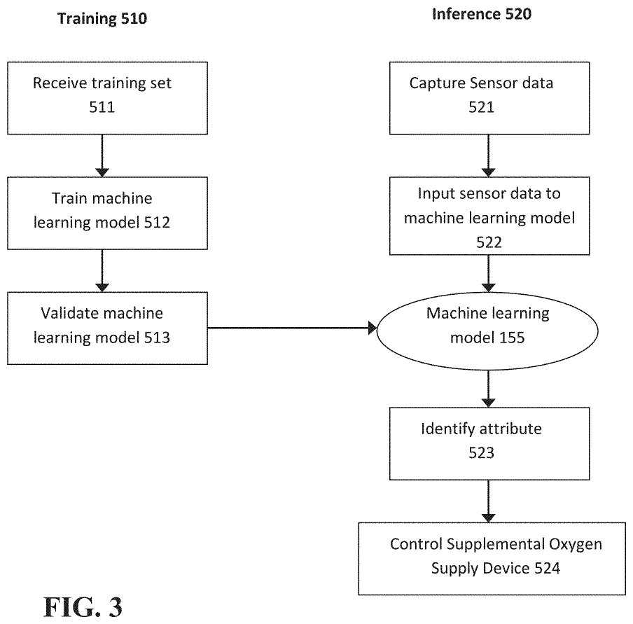

FIG. 3 is a flow diagram illustrating training and operation of a machine learning model (MLM 155), according to an embodiment. The process includes two main phases: training 510 the MLM 155 and inference (operation) 520 of the MLM 155. These will be illustrated using an example where the machine learning model learns to predict the required increase in supply of enriched oxygen gas based on historical data of increases in heart rate, motion, and or a decrease in barometric pressure. The following example will use the term “machine learning model” but it should be understood that this is meant to also include an ensemble of machine learning models.

A training module (not shown) performs training 510 of the MLM 155. In some embodiments, the MLM 155 is defined by an architecture with a certain number of layers and nodes, with biases and weighted connections (parameters) between the nodes. During training 510, the training module determines the values of parameters (e.g., weights and biases) of the MLM 155, based on a set of training samples.

The training module receives 511 a training set for training the machine learning model in a supervised manner. Training sets typically are historical data sets of inputs and corresponding responses by a supplemental oxygen supply device. The training set samples the operation of the supplemental oxygen supply device, preferably under a wide range of different conditions. FIG. 2A gives some examples of input data 310 that may be used for a training set. The corresponding responses are alterations of the supplemental oxygen supply device and subsequent observations after some time interval, such as the actual SpO₂% for a patient after an oxygen enriched gas increase in flow.

The following is an example of a training sample at 12:00 PM:
i. Heart Rate 60 bpm, increasing at 3 bpm/10 seconds
ii. Movement from prone to standing
iii. 95% SpO₂%
iv. 1,000 mb

After 5 minutes, the supplemental oxygen supply device 110 has increase the flow of oxygen enriched gas by 0.5 L/minute and at 12:05 PM the observed responses are the following:
i. Heart Rate 72 bpm
ii. Walking at 3 ft/second
iii. 92% SpO₂%
iv. 1,000 mb

After another 5 minutes, the supplemental oxygen supply device 110 again increases the flow of oxygen enriched gas by 0.5 L/minute and at 12:10 PM the observed responses are the following:
i. Heart Rate 72 bpm
ii. Walking at 3 ft/second
iii. 95% SpO₂%
iv. 1,000 mb

In this example, the increase in the flow of oxygen by 0.5 L/min is not sufficient to bring the SpO₂ to an acceptable level of 95% by 12:05. Only after a total increase of 1 L/min is effected will the patient's SpO₂ level reach the acceptable level. The MLM 155 can use this as an example to learn that when the patient is sitting, then begins to walk at 3 ft/second, causing a heart rate increase of 3 bpm/10 seconds, the flow of oxygen should be initially increased by 1 L/min-not 0.5 L/min.

In typical training 510, a training sample is presented as an input to the MLM 155, which then predicts an output for a particular attribute. The difference between the machine learning model's output and the known good output is used by the training module to adjust the values of the parameters (e.g., features, weights, or biases) in the MLM 155. This is repeated for many different training samples to improve the performance of the MLM 155 until the deviation between prediction and actual response is sufficiently reduced.

The training module typically also validates 513 the trained MLM 155 based on additional validation samples. The validation samples are applied to quantify the accuracy of the MLM 155. The validation sample set includes additional samples of inputs, known responses from the supplemental oxygen supply device 110, and subsequent SpO₂ measurements. The output of the MLM 155 can be compared to the known ground truth. To evaluate the quality of the machine learning model, different types of metrics can be used depending on the type of the model and response.

Classification refers to predicting what something is, for example if an image in a video feed is a person. To evaluate classification models, F1 score may be used. The F1 score is a measure of predictive accuracy of a machine learning model. The F1 score is calculated from the precision and recall of the machine learning model, where the precision is the number of correctly identified positive results divided by the number of all positive results, including those not identified correctly, and the recall is the number of correctly identified positive results divided by the number of all samples that should have been identified as positive.

Regression often refers to predicting quantity, for example, how much energy is consumed. To evaluate regression models, coefficient of determination, which is a statistical measure of how well the regression predictions approximate the real data points, may be used. However, these are merely examples. Other metrics can also be used. In one embodiment, the training module trains the machine learning model until the occurrence of a stopping condition, such as the metric indicating that the model is sufficiently accurate or that a number of training rounds having taken place.

Training 510 of the MLM 155 can occur off-line, as part of the initial development and deployment of system 100. Under this option, training samples from other patients (historical control information 145) can be used to train the MLM 155. This training data can be all available historical control information 145, or a portion of the historical control information 145 for other patients that are similarly situated such as, for example, being the same/similar age, same sex, same/similar respiratory issue/disease.

The trained MLM 155 is then deployed in the field. Once deployed, the MLM 155 can be continually trained 510 or updated. For example, the training module uses data captured in the field, during use of the supplemental oxygen supply device 110, to further train the MLM 155. The training 510 can occur within control system 150 and/or in an external database.

In operation 520, the MLM 155 uses the same inputs as input 522 to the MLM 155. The MLM 155 then predicts the corresponding response. In one approach, the MLM 155 calculates 523 a probability of possible different outcomes, for example the probability that a patient's SpO₂% will go below 90% after a decrease in air pressure and/or increased patient motion and/or increased patient heart rate. Based on the calculated probabilities, the MLM 155 identifies 523 which attribute is most likely. In a situation where there is not a clear cut winner, the MLM 155 may identify multiple attributes and ask the patient, or a third party, to verify.

Continuing the above example, the patient continues to walk, and gain altitude (thus reducing barometric pressure). The inputs to the MLM 155 are the following:
i. Heart Rate 75 bpm;
ii. Walking at 3 ft/second;
iii. 92% SpO₂%; and
iv. Reduction of 10 mb, to 990 mb, over 5 minutes

The MLM 155 predicts the following attributes 523:
i. Predicted increase in flow of oxygen enriched gas is greater than typical step-wise increase of just 0.5 L/min, therefore, MLM predicts an increase of 1.5 L/min increase in flow of oxygen enriched gas.

The controller 160 controls the supplemental oxygen supply device 524 by using the responses predicted by the MLM 155 to make informed decisions.

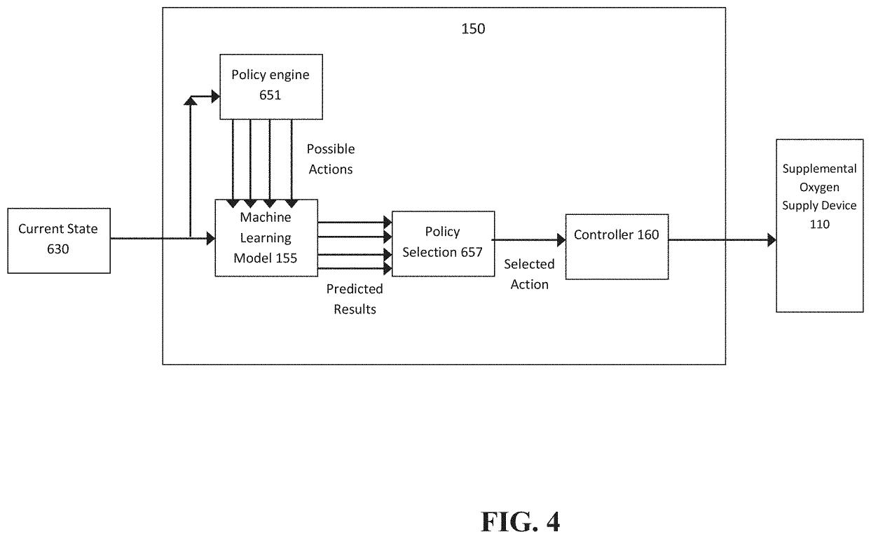

FIG. 4 is a block diagram of the control system 150 that uses the MLM 155 to evaluate different possible courses of action. In this example, the MLM 155 functions as a simulation of the supplemental oxygen supply device 110. The current state 630 of the patient's SpO₂ levels, heart rate, motion and barometric pressure are the inputs to the MLM 155. For example, the state might include the patient's SpO₂ level being 94%, heart rate of 65 bpm, walking at 1 ft/second and 1,000 mb. The control system 150 can take different courses of action to affect the patient's SpO₂ levels. For example, the control system 150 can increase the flow of oxygen enriched gas, reduce the flow of oxygen enriched gas, or maintain the currently provided flow of oxygen enriched gas.

A “policy” is a set of actions performed by the control system 150. In the above scenario, some example policies are as follows:
i. Policy 1: Increase flow of oxygen enriched gas by a step-wise increase amount because current inputs indicate a future SpO₂ level below acceptable range;
ii. Policy 2: Increase flow of oxygen enriched gas by an amount greater than step-wise increase because current inputs indicate a future SpO₂ level is below acceptable range;
iii. Policy 3: Reduce flow of oxygen enriched gas because current SpO₂ level is above acceptable range; and
iv. Policy 4: maintain flow of oxygen enriched gas because current SpO₂ level is within acceptable range.

The policies can be a set of logic and rules determined by domain experts. They can also be learned by the control system 150 itself using reinforcement learning techniques. At each time step, the control system 150 evaluates the possible actions that it can take and chooses the action that maximizes evaluation metrics. It does so by simulating the possible subsequent states that may occur as a result of the current action taken, then evaluates how valuable it is to be in those subsequent states. For example, a valuable state can be that the patient's new SpO₂ level is within an acceptable range in response to a larger that step-wise increase in the flow of oxygen enriched gas (for example, the valuable state can be the result of an increase in flow of 1.5 L/min based on the patient's input, as the control system 150 learns that three step wise increases of 0.5 L/min takes longer to increase SpO₂ levels).

Based on the current state 630, a policy engine 651 determines which polices might be applicable to the current state. This can be done using a rules-based approach, for example. The MLM 155 predicts the result of each policy. The different results are evaluated and a course of action is selected 657 and then carried out by the controller 160. A set of metrics is used to evaluate the policies. For example, if an acceptable SpO₂ level is 95% or greater, then a policy that results the patient's current SpO₂ level being below that level for too long is scored poorly. Policy selection 657 can also be reported to one or both of the historical data database 140 and the historical information database 144 in any suitable way, so that historical policies can be referred to and used.

Metrics can be defined to suit particular needs. For example, metrics to evaluate patients with various restrictive and/or obstructive lung conditions, diseases, and/or diagnoses. In one embodiment, the metrics can be defined to treat a a patient with chronic obstructive pulmonary disease (COPD) exhibiting asthma, which may have different metrics requires to manage COPD patients with different diagnoses or symptoms, such as bronchitis and/or emphysema, or patients with bronchitis and/or emphysema but not diagnosed with COPD. Metrics can also be defined for different time horizons. For example, a policy may be chosen to optimize for immediate gains, while another may be chosen to optimize for long-term benefits

To simulate subsequent states, the control system 150 uses the trained MLM 155. When underlying conditions (e.g. progress/diminishment of respiratory health of the patient) are changing, the MLM 155 can make predictions on what most likely will be observed as a result of actions taken. Based on these predictions, the control system 150 chooses a policy or action that most likely maximizes the metric of interest, being the SpO₂ level continuing to be within acceptable limits.

To decide which action to take from a state, the control system 150 may employ techniques of exploitation and exploration. Exploitation refers to utilizing known information. For example, a past sample shows that under certain conditions, a particular action was taken, and good results were achieved. The control system 150 may choose to exploit this information, and repeat this action if current conditions are similar to that of the past sample. Exploration refers to trying unexplored actions. With a pre-defined probability, the control system 150 may choose to try a new action. For example, 10% of the time, the control system may perform an action that it has not tried before but that may potentially achieve better results.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A method of controlling a supplemental oxygen supply device, the method comprising:
receiving data from one or more sensors of a patient device operably connected to a patient;
using the data as an input to a machine learning model that predicts whether or not a change to a supply amount of an oxygen enriched gas from the supplemental oxygen supply device is to be made; and
transmitting a control signal to the supplemental oxygen supply device based on the predicted change.

2. The method of claim 1, further comprising accessing historical data and using the historical data as additional input to the machine learning model.

3. The method of claim 2, wherein the historical data is stored data from the patient device selected from the group consisting of the patient's heart rate, motion of the patient, a patient's percentage of oxygen-saturated hemoglobin (SpO₂), barometric pressure of the patient and combinations thereof.

4. The method of claim 1, further comprising accessing historical information from other patients and using the historical information as an additional input to the machine learning model.

5. The method of claim 4, wherein the historical information is stored data from one or more additional patients selected from the group consisting of the patient's heart rate, motion of the patient, a patient's percentage of oxygen-saturated hemoglobin (SpO₂), barometric pressure of the patient and combinations thereof.

6. The method of claim 1, wherein the data received from the one or more sensors is selected from the group consisting of the patient's heart rate, motion of the patient, a patient's percentage of oxygen-saturated hemoglobin (SpO₂), barometric pressure of the patient and combinations thereof.

7. The method of claim 1, wherein the supplemental oxygen supply device receiving the control signal is an oxygen concentrator.

8. The method of claim 1, wherein the data from the patient device is received continuously.

9. The method of claim 1, wherein the supply of oxygen enriched gas is delivered in one of a pulse flow and a continuous flow.

10. The method of claim 1, wherein the supply of oxygen enriched gas is composed of at least about 50% oxygen or more, at least about 60% oxygen or more, at least about 70% oxygen or more, at least about 80% oxygen or more, at least about 85% oxygen or more, at least about 86% oxygen or more, at least about 87% oxygen or more, at least about 88% oxygen or more, at least about 89% oxygen or more, at least about 90% oxygen or more, at least about 91% oxygen or more, at least about 92% oxygen or more, at least about 93% oxygen or more, at least about 94% oxygen or more, at least about 95% oxygen or more, at least about 96% oxygen or more, at least about 97% oxygen or more, at least about 98% oxygen or more, at least about 99% oxygen or more.

11. The method of claim 1, further comprising transmitting an alert signal to a server.

12. The method of claim 11, wherein the server automatically transmits the alert signal to one or more mobile devices.

13. A system comprising:
an input module configured to receive data from one or more sensors of a patient device operably connected to a patient;
a machine learning model configured to receive the data as input and predict whether or not a change to a supply amount of an oxygen enriched gas from an supplemental oxygen supply device is to be made; and
a controller configured to control the supplemental oxygen supply device based on the predicted change.

14. The system of claim 13, wherein the input module is further configured to receive historical data and using the historical data as additional input to the machine learning model.

15. The system of claim 14, wherein the historical data is stored data from the patient device selected from the group consisting of the patient's heart rate, motion of the patient, a patient's percentage of oxygen-saturated hemoglobin (SpO₂), barometric pressure of the patient and combinations thereof.

16. The system of claim 13, wherein the input module is further configured to receive historical information from other patients and using the historical information as an additional input to the machine learning model.

17. The method of claim 16, wherein the historical information is stored data from one or more additional patients selected from the group consisting of the patient's heart rate, motion of the patient, a patient's percentage of oxygen-saturated hemoglobin (SpO₂), barometric pressure of the patient and combinations thereof.

18. The system of claim 13, wherein the data received from the one or more sensors is selected from the group consisting of the patient's heart rate, motion of the patient, a patient's percentage of oxygen-saturated hemoglobin (SpO₂), barometric pressure of the patient and combinations thereof.

19. The system of claim 13, wherein the supplemental oxygen supply device receiving the control signal is an oxygen concentrator.

20. The system of claim 13, wherein the supply of oxygen enriched gas is controlled for delivery in one of a pulse flow and a continuous flow.

21. The system of claim 13, wherein the supply of oxygen enriched gas is composed of at least about 50% oxygen or more, at least about 60% oxygen or more, at least about 70% oxygen or more, at least about 80% oxygen or more, at least about 85% oxygen or more, at least about 86% oxygen or more, at least about 87% oxygen or more, at least about 88% oxygen or more, at least about 89% oxygen or more, at least about 90% oxygen or more, at least about 91% oxygen or more, at least about 92% oxygen or more, at least about 93% oxygen or more, at least about 94% oxygen or more, at least about 95% oxygen or more, at least about 96% oxygen or more, at least about 97% oxygen or more, at least about 98% oxygen or more, at least about 99% oxygen or more.

22. The system of claim 13, wherein the controller is further configured to transmit an alert signal to a server.

23. The system of claim 22, wherein the server is configured to automatically transmit the alert signal to one or more mobile devices.
